(19) United States
(12) Patent Application Publication
(10) Pub. No.: US 2007/0082868 A1
(43) Pub. Date: Apr. 12, 2007

(54) Seaweed extract composition for treatment of diabetes and diabetic complications

(76) Inventor: Bruce Alan Daniels, Oklahoma City, OK (US)

Correspondence Address: INHOUSE CO., 280 COLORADO AVE., PALO ALTO, CA, 94301, US

(21) Appl. No.: 11/635,882

(22) Filed: Dec. 7, 2006

Related U.S. Application Data
(62) Division of application No. 10/841,101, filed on May 6, 2004.
Application No. 10/841,101 is a continuation-in-part of application No. 10/438,088, filed on May 14, 2003, now Pat. No. 7,022,682.
Application No. 10/438,088 is a continuation-in-part of application No. 10/320,309, filed on Dec. 16, 2002, now Pat. No. 6,797,705.

(51) Int. Cl. A61K 31/715; A61K 36/02

(52) U.S. Cl. 514/54; 424/195.17

(57) ABSTRACT

A method and composition is described for prevention or treatment of Type II Diabetes Mellitus and its complications comprising the step of administering to a mammal an amount of seaweed extract in effective doses. The seaweed is selected from the group consisting of brown algae, red algae and green algae, wherein the seaweed extract contains a polysaccharide. Optionally, the polysaccharide is Rhamnose, in approximately 63-78 mole %. The seaweed extract optionally also contains approximately 6.5-9.2 mole % Xylose. Optionally, Rhamnose and Xylose are present in the seaweed extract in amounts in a ratio of approximately 12 Rhamnose to 1 Xylose and 8 Rhamnose to 1 Xylose. Preferably, the Rhamnose (algopolysaccharide) is sulfinated. Optionally, the seaweed extract is mixed with food stuffs such as cereals, bread, drinks, health bars, juices, concentrates, canned food, ice cream, water, staple goods such as wheat, corn, barley, and oat in any form, or taste maskers such as sugar or ascorbic acid.

### CROSS-REFERENCE

This application is a divisional of application Ser. No. 10/841,101, filed May 6, 2004, which is a continuation-in part of application Ser. No. 10/438,088, filed May 14, 2003, entitled: “Novel Composition and Method for the Treatment of Diabetes”, which is a continuation-in-part of application Ser. No. 10/320,309, filed Dec. 16, 2002, entitled: “Rhamnan Sulphate Composition for treatment of Endothelial Dysfunction” all of which is hereby incorporated by reference.

### FIELD

This invention relates to a pharmacological composition and method that provides for regression of neuropathies associated with but not limited to diabetes, preservation of renal function in diabetic states and other vasculopathic states. This composition is preferably used for patients susceptible to or suffering from diabetes, an endothelial dysfunction disorder or disease, an inflammatory state resulting in endothelial and vascular dysfunction, and more particularly, but not by way of limitation, to a formulation with enhanced absorption characteristics for preventing and/or treating, type II diabetes mellitus, and its complications, without appreciably increasing the patient's risk of hemorrhaging, either internal or as a result of an external injury.

### BACKGROUND

Type II Diabetes Mellitus and other diseases resulting from endothelial dysfunction, and their associated complications are a principal cause of disabilities and deaths of individuals in the world. For example, in recent years more than 700,000 deaths have occurred annually in the United States alone as a result of coronary artery disease, and many more patients have been hospitalized for unstable angina, acute myocardial infarction, and congestive heart failure, which occur in greater than 70% of patients with diabetes as the disease progresses. Additionally, diabetes is the most common cause of chronic renal insufficiency and renal failure in industrialized societies and a major cause of blindness and limb loss due to leg ischemia.

There has been significant and extensive research for effective long-term treatment for diabetes. However, present treatments for such disorders are partial treatments such as administration of insulin, and oral hypoglycemic agents. These treatments have serious shortcomings in long-term effectiveness. The use of these treatments does not resolve the spectrum of molecular and physiologic abnormalities attendant to the diabetes processes.

Diabetes is a medical condition associated with low eNOS activity, and high superoxide and other free radicals being generated in association with cellular hyperglycemia. These abnormalities result in alterations and defects in cellular matrix composition and cellular function in a variety of cell types including the pancreas, liver, kidneys, endothelium and the cardiovascular system.

Currently available treatments have to date been only partially effective for favorable long-term results. None of these treatments have been designed to maintain, improve, or restores cellular function of the cell matrix of these cited organs and tissues by impacting the cell matrix and control of the generation of nitric oxide, superoxide, and other free radicals.

The focus of current treatment methods is to react to potentially immediate danger to one's life. These treatments and their shortcomings include;
Insulin injections: Shortcoming: hypoglycemia, inconvenience, and discomfort of administration.
Diet: Shortcoming: hard to follow.
Oral Medication: Metformin, Glucophage, Shortcoming: for severe hyperglycemia it is insufficient and does not treat the other dysmetabolic effects of the disease.
Kidney-Pancreas transplant: Shortcoming: Can't find donors.

None of these treatment methods is directed towards the underlying disease processes, the molecular causes of the disease or disorders, the effects of the hyperglycemia on the basal molecular organization and properties of the cells, or towards restoring the structure and function of the blood vessels and other cell types to levels that reduce or eliminate the danger posed by diabetes. There is no treatment designed to reduce the level of free radical generation, of cell matrix re-organization, cell membrane composition, of clotting activity or preservation of vascular thromboresistance.

In view of the foregoing, there is a significant need for a pharmacological composition and method that is directed towards treating the underlying diabetes disease process, and towards preserving and restoring the structure and improving the functions of the various cell types involved with the diabetic processes and in particular the function-structure properties of the endothelium which lines the entire cardiovascular system and which is the interface of the blood with the vascular system and a major determinant of effective renal function.

It is an objective of the present invention to provide a treatment, which is directed to preventing and minimizing dysfunctional atomic and molecular interactions within the human cellular matrix or cellular environment, which lead to diabetes and its myriad complications.

It is another objective of the present invention to provide a treatment that is directed to retarding adverse consequences of free radicals generated in human cellular matrix. It is also another objective of the present invention to stimulate an increased production of nitric oxide within human cellular matrix or cellular environment and to decrease production and effects of superoxide and other free radicals throughout the course of the disease.

It is yet another objective of the present invention to effect prevention and treatment of cardiovascular diseases, which are the greatest source of morbidity and mortality attending diabetes, and in particular cell surface based thrombosis, without appreciably increasing blood anticoagulation activity in patients.

### SUMMARY

A method and composition is described for prevention or treatment of Type II Diabetes Mellitus and its complications comprising the step of administering to a mammal an amount of seaweed extract in effective doses. The seaweed is selected from the group consisting of brown algae, red algae and green algae, wherein the seaweed extract contains a polysaccharide. Optionally, the polysaccharide is Rhamnose, in approximately 63-78 mole %. The seaweed extract optionally also contains approximately 6.5-9.2 mole % Xylose. Optionally, Rhamnose and Xylose are present in the seaweed extract in amounts in a ratio of approximately 12 Rhamnose to 1 Xylose and 8 Rhamnose to 1 Xylose. Preferably, the Rhamnose (algopolysaccharide) is sulfinated.

A Type II Diabetes complication is selected from the group comprising neuropathy, nephropathy, arteriosclerosis, and proteinuria.

The composition and method of the invention further comprising the co-administration of a nitrogen donor or nitric oxide donor together with the seaweed extract, wherein the nitrogen donor is selected from the group consisting of L-Arginine and Lysine.

An advantage of the method and composition of the invention is that it possesses extremely potent anti-thrombotic activity and other inhibitory effects on cell surface coagulation assembly and activity for thrombus inhibition. It is well understood that accelerated cardiovascular arteriosclerosis, hypertension, diabetic nephropathy, and congestive heart failure are a few of the long-term complications of the diabetic processes. Decreased eNOS activity, increased free radical generation, increased thrombogenic activity and therefore thrombotic and ischemic complications accompany each of these disease states.

Another advantage of the described composition is that there is less peptide residual in extracting the composition from plant cells as compared to heparin from animal cells. Hence, it is less allergic reaction prone and has fewer immunogenic properties.

Yet another advantage is that since seaweed extract is from plant cells, it has no potential for the transmission of potentially lethal and serious prion diseases such as mad cow disease.

Another advantage is that seaweed extract has no potential for activating Platelet Factor IV and resulting in immune complex destruction of platelets as seen with heparin administration.

Finally, another advantage of seaweed extract is that it is a functional substitute for heparin in applications requiring systemic anticoagulant activity such as dialysis, bypass surgery, and polymer tube coatings and devices for use in mammals and humans.

It is also another advantage of the present invention in that it reduces C-reactive protein levels in the plasma, which are recognized to predict the future occurrence of diabetes and vascular disease.

It is another advantage of the present invention in that it preserves healthy cardiovascular function including blood vessels, by lowering LDL and/or increasing HDL level in the plasma.

### BRIEF DESCRIPTION OF DRAWINGS

FIG. 1 is a graph comparing a control group to Heparin and different doses of sulfinated seaweed extract, Rhamnan Sulphate.

FIG. 2 is a graph comparing a control group to different doses of sulfinated seaweed extract, Rhamnan Sulphate, and L-Arginine.

FIG. 3 is a chemical representation of sulfinated seaweed extract, Rhamnan Sulphate (polysaccharide or algopolysaccharide), and Rhamnan Sulfate salt of Arginine.

FIG. 4 shows the effect of an optional embodiment of the composition of the invention on C Reactive Protein (CRP) levels, in healthy, hypertensive, and peripheral arterial occlusive human subjects.

FIG. 5 shows the effect of an optional embodiment of the composition of the invention on High Density Lipoprotein (HDL) levels, in healthy, hypertensive, and peripheral arterial occlusive human subjects.

FIG. 6 shows the effect of an optional embodiment of the composition of the invention on Low Density Lipoprotein (LDL) levels, in healthy, hypertensive, and peripheral arterial occlusive human subjects.

FIG. 7 shows the effect of an optional embodiment of the composition of the invention on Triglyceride levels, in healthy, hypertensive, and peripheral arterial occlusive human subjects.

FIGS. 8, 9, and 10 show the effect of an optional embodiment of the composition of the invention on systolic and diastolic blood pressure levels, in healthy, hypertensive, and peripheral arterial occlusive human subjects.

### DETAILED DESCRIPTION

As has been previously described by the inventor, a cellular environment (cellular matrix, gel matrix, or gel) composed of charged polymers-highly charged peptide-water polymers, such as heparin-arginine-water, is responsible for controlling the structure and ultimately the function of human cells within this cellular environment. As the human blood vessel is only one cell thick, it too operates within this charged polymer-highly charged peptide-water environment. Thus, this charged polymer-arginine-water environment impacts such important functions of the cells by effecting protein distribution and functionality, cell signaling processes, genetic or DNA-RNA transcription regulation, and the physical/chemical properties of cells, including blood vessel wall cells. The inventor, in U.S. Pat. Nos. 6,255,296 and 6,495,530, outlines the fact that a disruption in the structure of the cellular environment, and in particular the endothelial cells, is the proximate cause of diseases such as cardiovascular disease. The specifications of those patents are hereby incorporated by reference herein. While not bound to a particular mechanism of operation, the inventor's conception is that the loss of heparin sulfate causes a reduction in Nitric Oxide formation.

It should be noted that heparins or heparin domains within these polymer structures are members of the group commonly referred to as endogenous heparans. Exogenous heparans, including heparin, have functions, which protect the endogenous heparans.

The present invention is directed to a composition for treatment of Type II Diabetes, and retardation of related diseases or complications such as neuropathy, nephropathy, and proteinuria in patients with Type II diabetes. Retardation is defined as to slow down the progression or development of Type II Diabetes and related complications.

In accordance with the invention, a patient susceptible to or suffering from Type II Diabetes or related diseases or complications is treated with a dose or amount of substance characterized as a seaweed extract. Additionally, a healthy individual, who over time would likely develop Type II Diabetes, is also treated with a dose or amount of the composition of the present invention.

The definition of the term extract used in seaweed extract is not limited to the extraction method in Example 1, rather the term is used broadly to include crushing the seaweed and mixing with water or other ingredients; chopping, grinding, mincing, or forming a paste of the seaweed, processing the seaweed into a dry powder, extruding, fermenting, or any other process by which the polysaccharide, such as Rhamnose or other polysaccharides remain in the extract.

The seaweed is preferably selected from the group consisting of brown algae and green algae. Brown algae is selected from the group consisting of Fucus vesiculosus, Laminaria brasiliensis, or Ascophylum nodosum. Green algae is selected from the group consisting of Monostroma nitidium, Monostroma zosteticola, Monostroma angicava, Monostroma lattlsglmum, Monostroma pulchrum, Monostroma fusem, Monostroma grevillei, Entoromorpha compressa, Ulva arasakii, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

An amount of seaweed extract containing Rhamnose or other polysaccharide, such that it is sulfinated either synthetically or by natural means such as metabolism in a mammal, and which is useful and effective in retardation of Type II Diabetes, is defined primarily by clinical response in a patient, and ranges from about an equivalent of 2,000 IU to 200,000 IU heparin activity. A more preferred range of an effective amount of seaweed extract is equivalent to between about 5,000 to 20,000 IU heparin activity. A most preferred range of an effective amount of seaweed extract is equivalent to between 8,000 IU and 12,000 IU heparin activity.

Sulfination of a polysaccharide is defined as attaching a sulfate group to the polysaccharide to form a sulfated moiety of the polysaccharide. For example, forming Rhamnan Sulfate from Rhamnose, or Xylose Sulfate from xylose. Rhamnan Sulfate backbone can be substituted at the 2-, 3- or 4-positions with minor amounts (less than 10% in total) of galacturoinc acid, xylose and arabinose.

When absorbed into the charged polymer-highly charged peptide-water matrix, the polysaccharide in the seaweed extract acts as a heparan and protects and reinforces structure and roles of endogenous heparans. Whatever the mechanism, the sulfinated polysaccharide in the seaweed extract has a potent effect to bind free radicals preventing damages to multiple cell types including pancreatic cells and endothelial cells.

Localization of administered heparin or heparin analogues to cell surfaces (e.g. endothelial surfaces) by oral administration inhibits thrombotic activity within and on artery and blood vessel surfaces without the inhibition of plasma clotting factors seen with currently available anticoagulants.

The sulfinated polysaccharide in the seaweed extract is characterized such that it should be administered in an amount sufficient to bind free radicals preventing damages to pancreatic cells and endothelial cells, while not appreciably increasing the patient's risk of internal or external hemorrhaging and effectively maintaining integrity and functionality of the cellular membranes and surrounding environments of the endothelial cells.

The inventor recognizes as integral to the invention, that cell surface based anti-thrombotic activity is distinctly different from plasma anti-coagulation. The invention achieves cell based anti-thrombotic activity without the inhibition of plasma anticoagulant factors. Thus, the invention avoids the risks of spontaneous hemorrhage or excessive bleeding due to vessel injury attendant to plasma anticoagulation.

Effective doses of the seaweed extract vary with the particular individual's condition and the method of administration. For example, it is noticed that subcutaneous injection of heparin results in greater concentration in the cellular and membrane domains than intravenous injection, and it is the inventor's observation that oral heparan sulphates localizes almost exclusively to cell surface membranes, especially the endothelium. Thus, the preferred method of administration of the seaweed extract for the present invention is through the oral route, while the least preferred method is via intravenous injection.

The seaweed extract is optionally formulated for oral, sublingual, subcutaneous, intravenous, transdermal or rectal administrations in dosages and in admixture with pharmaceutical excipients or vehicles including implantation or controlled-release devices. For example, the seaweed extract is optionally dispersed in a physiologically acceptable, non-toxic liquid vehicle, such as water.

Alternatively, the seaweed extract can be given in tablet, capsule, powder, granules, coated tablet form, or mixed with various food stuffs such as; cereals, bread, drinks, health bars, juices, concentrates, canned food, ice cream, water, staple goods such as wheat, corn, barley, and oat in any form, processed or not, or taste maskers such as sugar or ascorbic acid, or other functional foods. The seaweed extract, including its sulfinated polysaccharide may be mixed with conventional pharmaceutical auxiliaries, such as binders, fillers, preservatives, tablet disintegrators, flow regulators, plasticizers, wetting agents, dispersants, emulsifiers, solvents, retarding agents and/or anti-oxidants. It is also optionally contained or formed into a complex with lipids in various formulations and molecular arrangements.

An efficiently operating homeostatic system is crucial to cellular function within mammalian organisms. In a healthy state, there is formed a gel matrix of heparin, highly charged peptide and water polymers, which houses a plurality of other molecules by accommodating dynamic binding of and release of such molecules without reaching concentration levels which destroy the gel structure and its regulatory functionalities.

Long chain charged polymer strands are an organizing determinant for membranes, proteins, receptors, ion channels, cell organelles, nuclear membranes, membrane pores, and other complex cellular constituents. The polymers and highly charged amino acids such as Arginine organize water into arenas for confining bilipid layer membranes, for example, creating cell turgor and form and limiting hydrolytic properties of water on other molecular structures.

A healthy gel matrix is formed from endogenous charged polymers, endogenous arginine and water. An unhealthy state of a gel matrix has some of the highly charged peptides molecules cleaved out of the gel. Likewise, charged polymers have been removed from the gel. There are thus created gaps between charged polymers into which other molecules can embed or pass through.

The healthy gel structure has a conformation that preferentially supports interaction and binding of foreign molecules. The capacity to accommodate intrusions of such molecules before the gel structure collapses and loses its functionality is an important characteristic of the gel system.

The permeability of the membranes thus allows macromolecules or cells to enter and traverse the gel. For example, cholesterol, clotting factors and water traverse the gel reaching a bilipid layer, or other sub endothelial locations. In addition, ionic strength, flow stress, heat, osmotic pressure or other forms of energy transfer to the gel can deteriorate the properties of the gel as described above.

These intrusions result in a displacement of arginine and decreased generation of nitric oxide as an additional effect. Intrusions limit the binding capacity of the heparin for arginine and other molecules within the gel.

Further, while not being bound to a mechanism of operation, LDL receptors on pancreatic islet cells take up LDL, which leads to inflammation and tissue factor production (analogous to plaque in arteries). The sulfinated polysaccharide in seaweed extract is believed to suppress both the inflammation and tissue factor-induced coagulation in pancreatic cell membranes and cell matrix just as it does in endothelial and artery cell matrix. The sulfinated seaweed extract therefore prevents the progressive islet cell damage and insulinopenia associated with Type I and Type II diabetes.

In order to reverse this disruption of the gel matrix caused by the removal of arginine and/or heparin, the present invention employs the polysaccharides in seaweed extract (algopolysaccharides) to maintain and rejuvenate the gel matrix and its functionality. These algopolysaccharides are selected from the group consisting of; Rhamnose, Xylose, Galactose, and Mannose. In this regard, the present invention utilizes the polysaccharides in seaweed extract, which is particularly high in Rhamnose, to give optimal pore closure and stabilization, and number and distribution of binding sites, wherein signaling, anti-proliferation, cell surface anti-thrombotic, and anti-inflammatory effects are maintained. Thus, the homeostasis-promoting functionalities of heparin, arginine, and charged polymer-highly charged peptide-water gel matrix, resultant from the herein-described composition, retard continuous and accumulative change and injury to cellular domains. By this retarding effect, cholesterol accumulations generally referred to as “arterial plaques” are minimized.

Administration of seaweed extract also leads to increased lipoprotein and lipase release and tissue factor pathway inhibitor release, with beneficial effects on plaque stability, growth, rupture, and regression.

Addition of a sulfated Rhamnose polysaccharide, Rhamnan Sulfate, to the gel system protects the functionality of both heparin and the arginine in the gel matrix. In the extragellular medium, the ability of heparin to bind and quiesce molecules is augmented by simultaneous addition of Rhamnan Sulphate, wherein Rhamnan Sulphate binds to extragellular potentially-intruding molecules, thus allowing existing gellular charged polymers to associate with gellular arginine.

Nitric oxide produced from arginine is an important physiological mediator. The enzyme responsible for nitric oxide production, nitric oxide synthase, requires CA++ and Calmodulin. The functionality of the heparin-arginine gel includes its binding and regulation of CA++ and Calmodulin. By regulating Calmodulin activity, the effects of Rhamnan Sulphate on the charged polymer-arginine gel regulates nitric oxide synthase activity responsible for nitric oxide production.

Arginine acts as a nitrogen donor for the production of Nitric Oxide. However, it is understood that lysine is optionally used instead of Arginine as a nitrogen donor. A larger dose of lysine may be required to be as effective as a smaller dose of arginine. Other nitric oxide donors also may be optionally used.

The binding of water, small anions and cations within the charged polymer-arginine-water gel is facilitated by pi-bonding properties inherent in the saccharide ring structure within the charged polymers. Changes in the shared electron density and electrical charge variation regulated the state of solvation and conformation of the gel polymers. Thus, small anion and cation binding induces changes in the state of solvation, changes in catalytic and hydrolytic properties of water, and changes in capacity of the gel to bind water and other molecules. Low to high molecular weight Rhamnose derived from seaweed extract, preferably having a high degree of sulfation, is preferably used.

Endothelial cell injury and pancreatic cell injury occur from free radicals. Heparin binds super oxide dismutase, which absorbs high-energy electrons and deactivates free radicals. Rhamnan Sulphate, heparin, and nitric oxide bind free radicals preventing damages to multiple cell types including pancreatic cells and endothelial cells.

Type II Diabetes is in part due to free radical injury to pancreatic cells. Rhamnan Sulphate, heparin, super oxide dismutase and nitric oxide all attack and neutralize free radicals, therefore, diseases associated with cellular injury from free radicals are effectively treated and prevented by the present invention. Also, Rhamnan Sulphate aids in the reconstruction of damaged tissue by promoting the production of endogenous heparin, which then forms a complex with and removes extra cellular matrix protein accumulations, e.g. fibronectin with consequent reversal or minimization of organ hypertrophy states. Rhamnan Sulphate enhances regeneration of the endothelium following an injury to an endothelium surface.

### EXAMPLE 1

### Extraction Method

Dried green Algae (Monostroma Nitidium) was swollen in 10 Vols. Of water at room temperature for one hour. Thereafter the swollen green algae was ground and refluxed for two (2) hours in a boiling water bath. The water extract was centrifuged (4500 g) for 30 minutes, and the water-soluble polysaccharide in the non-dialyzable fraction was obtained by lyophilization.

The crude polysaccharide was dissolved in water and was applied to a column (2.4×100 cm) of DEAE-cellulose (Whatman DE-52). Starch or neutral polysaccharides were removed by continuous water elution until the sample was completely free as determined by phenol-sulfuric acid detection. Afterwards, acid polysaccharide was fractionated by stepwise alteration of the ionic strength of KCL at 0.5. 0.7 and 2.0 M, and then each fraction was desalted and freeze dried. The 0.5 M KCL fraction (major fraction) successive purification procedures were performed by gel filtration chromatography on a Toyopearl HW-65 (fine) column (1.2×100 cm). The sample was eluted with water at a flow rate of 0.4 ml/min. The major fraction was collected and freeze dried. These procedures or variations of them for extraction of complex polysaccharides are well known.

Composition Analysis of Seaweed Extract

Glycosyl Composition (Polysaccharide Composition)

Glycosyl composition analysis was performed by combined gas chromatography/mass spectrometry (GC/MS) of the per-O-trimethylsilyl (TMS) derivatives of the monosaccharide methyl glycosides produced from the sample by acidic methanolysis.

Methyl glycosides were first prepared from dry sample by methanolysis in 1 M HCl in methanol at 80° C. (18-22 hours), followed by re-N-acetylation with pyridine and acetic anhydride in methanol (for detection of amino sugars). The samples were then per-O-trimethylsilylated by treatment with Tri-Sil (Pierce) at 80° C. (0.5 hours). These procedures were carried out as previously described (Methods Enzymol. 230:1-15; York W. S., Darvill, A. G., McNeil, M., Stevenson, T. T., and Albersheim, P. (1985) Methods Enzymol. 118:3-40). GC/MS analysis of the TMS methyl glycosides was performed on an HP 5890 GC interfaced to a 5970 MSD, using a All Tech EC-1 fused silica capillary column (30 m×0.25 mm ID).

TABLE 1
Glycosyl Composition Analysis (Commercial Batches)

| Glycosyl residue | Mass (μg) | Mole %¹ |
|---|---|---|
| Rhamnose (Rha) | 657.4 | 69.6 |
| Fucose (Fuc) | n.d. | n.d. |
| Xylose (Xyl) | 57.4 | 6.6 |
| Glucuronic acid (GlcA) | 68.7 | 6.2 |
| Galaturonic acid (GalA) | n.d. | n.d. |
| Mannose (Man) | 20.2 | 2.0 |
| Galactose (Gal) | 33.2 | 3.2 |
| Glucose (Glc) | 128.8 | 12.4 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 888.9 | 63.1 |
| Fucose (Fuc) | n.d. | n.d. |
| Xylose (Xyl) | 102.0 | 7.9 |
| Glucuronic acid (GlcA) | 78.7 | 4.7 |
| Galaturonic acid (GalA) | n.d. | n.d. |
| Mannose (Man) | 47.2 | 3.1 |
| Galactose (Gal) | 68.4 | 4.4 |
| Glucose (Glc) | 260.5 | 16.8 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 907.2 | 70.2 |
| Fucose (Fuc) | n.d. | n.d. |
| Xylose (Xyl) | 80.2 | 6.8 |
| Glucuronic acid (GlcA) | 74.6 | 4.9 |
| Galaturonic acid (GalA) | n.d. | n.d. |
| Mannose (Man) | 29.7 | 2.1 |
| Galactose (Gal) | 41.1 | 2.9 |
| Glucose (Glc) | 186.2 | 13.1 |
| N-acetyl glucosamine (GlcNAc) | n.d. | n.d. |
| 3-deoxy-d-manno-2-octulosonic acid (Kdo) | n.d. | n.d. |
| Arabinose (Ara) | Trace | |
| Rhamnose (Rha) | 1010.9 | 66.8 |
| Fucose (Fuc) | n.d. | n.d. |
| Xylose (Xyl) | 98.2 | 7.1 |
| Glucuronic acid (GlcA) | 97.5 | 5.4 |
| Galaturonic acid (GalA) | n.d. | n.d. |
| Mannose (Man) | 42.7 | 2.6 |
| Galactose (Gal) | 71.1 | 4.3 |
| Glucose (Glc) | 230.2 | 13.8 |
| N-acetyl glucosamine (GlcNAc) | n.d. | n.d. |
| 3-deoxy-d-manno-2-octulosonic acid (Kdo) | n.d. | n.d. |
| Rhamnose (Rha) | 108.8 | 77.3 |
| Xylose (Xyl) | 11.6 | 9.0 |
| Glucuronic acid (GlcA) | 5.5 | 3.3 |
| Mannose (Man) | 2.3 | 1.5 |
| Galactose (Gal) | 5.3 | 3.4 |
| Glucose (Glc) | 10.5 | 5.5 |
| Rhamnose (Rha) | 129.0 | 78.1 |
| Xylose (Xyl) | 12.0 | 8.0 |
| Glucuronic acid (GlcA) | 5.7 | 2.9 |
| Mannose (Man) | 2.9 | 1.6 |
| Galactose (Gal) | 6.1 | 3.4 |
| Glucose (Glc) | 13.2 | 6.0 |
| Rhamnose (Rha) | 216.6 | 74.7 |
| Xylose (Xyl) | 24.5 | 9.2 |
| Glucuronic acid (GlcA) | 13.9 | 4.0 |
| Mannose (Man) | 4.6 | 1.4 |
| Galactose (Gal) | 9.6 | 3.0 |
| Glucose (Glc) | 30.3 | 7.7 |

Bench top analysis of seaweed extracts yielded between approximately 90% to 97% Rhamnose and between approximately 0% and 4.5% Xylose. The seaweed extract contains between approximately 63 mole % to 78 mole % Rhamnose. The seaweed extract contains between approximately 6.5 mole % to 9.2 mole % Xylose. The Rhamnose and Xylose are present in the seaweed extract in amounts between a ratio of approximately 12 Rhamnose to 1 Xylose and approximately 8 Rhamnose to 1 Xylose.

### EXAMPLE 2

### In Vivo Studies

Polysaccharide from seaweed extract, Rhamnan Sulphate, was prepared as described in Example 1 and was tested for cell surface anti-thrombotic activity, as described below. Rhamnan sulphate was dissolved in water at concentrations 20, 10 and 5 mg/ml. For experiments utilizing L-Arginine, L-Arginine capsules were opened and contents were dissolved in water at 300 mg/ml for 4 hr studies and 150 mg/ml for the 28 day study. Rhamnan sulphate-arginine complex (RS-LR), where L-Arginine is covalently bound to Rhamnan Sulphate to form a physiologically acceptable salt of Rhamnan Sulphate, was dissolved in water at concentrations of 20, 10, 3 and 0.3 mg/ml. Bovine ung unfractionated heparin, 150 units/mg, Lot No ZX320, was obtained from Upjohn Ltd. Heparin was dissolved in water at a concentration of 20 mg/ml.

One hundred and two male Wistar rats, weighing 312±64 g (±SD), were handled and housed according to the Principles of Animal Care set out by the Canadian Federation of Biological Societies. The animals were fasted overnight prior to treatment and were anaesthetized with barbital and methoxyflurane for experimental procedures.

Rhamnan sulphate was administered to rats at 7.5, 4 and 2 mg/kg with 5, 20, and 5 rats/group respectively. Rhamnan sulphate (7.5 mg/kg) plus arginine (112.5 mg/kg) was administered to 5 rats. Rhamnan sulphate-arginine complex was administered to rats at 4, 1 and 0.1 mg/kg with 20 rats/group. The rhamnan-arginine complex was weighed fresh daily. All of the Groups and the administered compounds are shown in Table 1. Six to 8 rats were treated per day. A stomach tube was filled with 0.2 ml saline followed by 0.09-0.18 ml of the rhamnan sulphate solutions or 0.1 ml of arginine solution depending on rat weight. Thus when the stomach tube was placed in the stomach the drugs were first introduced into the stomach followed by saline to give a total volume of approximately 0.4 ml. In the heparin alone group, heparin was administered in a volume of 0.1-0.2 ml followed by 0.2 ml saline. Control group was saline alone.

TABLE 2

| | |
|---|---|
| Group 1 | No Treatment |
| Group 2 | Heparin alone at 7.5 mg/kg |
| Group 3 | Rhamnan Sulphate alone at 7.5 mg/kg |
| Group 4 | Rhamnan Sulphate alone at 4 mg/kg |
| Group 5 | Rhamnan Sulphate alone at 2 mg/kg |
| Group 6 | Rhamnan Sulphate 7.5 mg/kg + L-Arginine |
| | 112 mg/Kg |
| Group 7 | Salt of Rhamnan Sulphate 4 mg/kg − L-Arginine |
| Group 8 | Salt of Rhamnan Sulphate 1 mg/kg − L-Arginine |
| Group 9 | Salt of Rhamnan Sulphate 0.1 mg/kg − L-Arginine |

Thrombosis Test

The thrombosis test was performed by a modification of the procedure by Blake et al. For animals exposed to treatment for 4 h, a thrombus was initiated in the right jugular vein by application of 10% formalin in 65% methanol to the exposed adventitial surface. Immediately following, drugs were introduced into the stomach by stomach tube. At 4 h after thrombus initiation animals were again deeply anaesthetized and first examined for any external signs of bleeding. The jugular vein was exposed and examined for the presence of a plug using a cotton pledget. The clot was scored as +(hard clot) if the vessel is blocked and remained blocked despite examination with a cotton pledget. The clot was scored as +/−(soft clot) if the vessel appeared completely blocked on first examination and then opened as it was examined. The thrombus was scored as −(negative) if blood was seen to flow freely in the vessel.

Collection of Blood and Blood vessels.

Immediately after examination of the jugular vein, a laparotomy was performed and a blood sample of approximately 10 ml (9 parts blood to 1 part 3.8% sodium citrate) was taken from the abdominal aorta. Plasma was prepared. As a source of endothelium, the thoracic aorta or vena cava was removed and placed in saline. Each animal was examined for signs of internal hemorrhage and the time when blood clotted in the body cavity was recorded.

Harvesting of Endothelium

Endothelium was removed from blood vessels according to the method of Hiebert and Jaques. The vessels were slit open, pinned to dental wax lumen side up, and rinsed in Locke's solution. Cellulose acetate paper was applied to the lumenal surface and when lifted, endothelium was removed. The length and width of the imprint were measured to the nearest mm.

Determination of Heparin-Like Compounds Within Endothelium

Cellulose acetate paper was removed from endothelium by dissolving in cold acetone followed by centrifuging and discarding the supernatant. The precipitates were further processed by digestion with pronase (10 μl of 40 mg/ml in Tris buffer). Samples were then centrifuged at 10,000 rpm for 10 min, supernatant was collected and the precipitate washed twice with 100 uL 26.8% NaCl which was added to the supernatant. Glycoseaminoglycans (GAGs) were precipitated from the supernatant with five volumes of methanol and the precipitate dried. Agarose gel electrophoresis was used to identify and measure rhamnan sulphate in endothelial extracts by previously published methods. The dried powders, dissolved in suitable volumes of water, were applied to agarose gel slides along with the administered rhamnan sulphate used as a reference. Following electrophoresis, gels were fixed in 0.1% hexadecyltrimethylammonium bromide and air dried. Slides were stained with 0.04% toluidine blue in 80% acetone and background color was removed with 1% acetic acid. Heparin was identified by electrophoretic migration as compared to reference material and amounts determined by densitometry.

Statistical Analysis

Thrombosis data is expressed as a percentage with 95% confidence intervals. X² test for differences between proportions was used to compare the total thrombotic incidence and incidence of hard clots between groups. Other data is expressed as mean±SE. A one-way ANOVA with Tukeys post hoc test was used to compare the differences between groups when plasma coagulation tests and heparin-like concentrations in urine were examined.

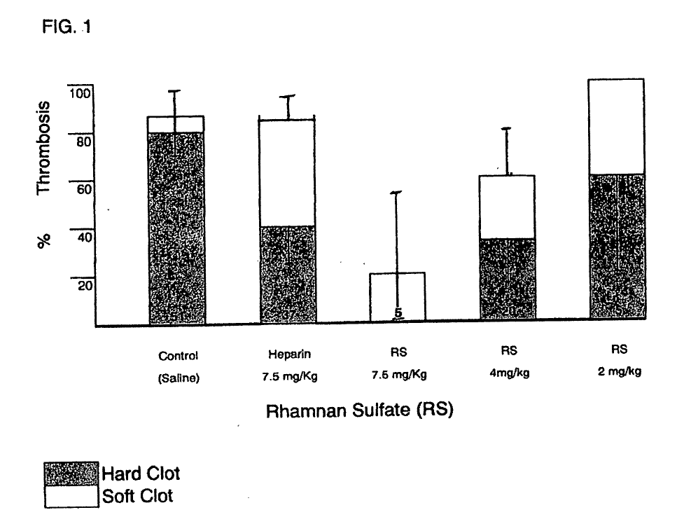

As shown in FIG. 1 an anti-thrombotic effect was observed with all oral doses of rhamnan sulphate alone, except 2 mg/kg. As well an anti-thrombotic effect was seen when arginine was added along with rhamnan sulphate or when rhamnan sulphate was complexed to arginine. At 2 mg/kg rhamnan sulphate there was a trend towards a significant reduction in hard clots versus controls although this did not reach significance. A dose response was evident with both rhamnan sulphate alone or when rhamnan sulphate was complexed to arginine. The rhamnan sulphate arginine complex was a significantly more effective anti-thrombotic agent than rhamnan sulphate alone as shown by a decrease in incidence of hard clots when comparing the compounds at 4 mg/kg. Further, the incidence of hard clots and total thrombotic incidence was less for the rhamnan sulphate arginine complex at 1 mg/kg versus rhamnan sulphate alone at 2 mg/kg.

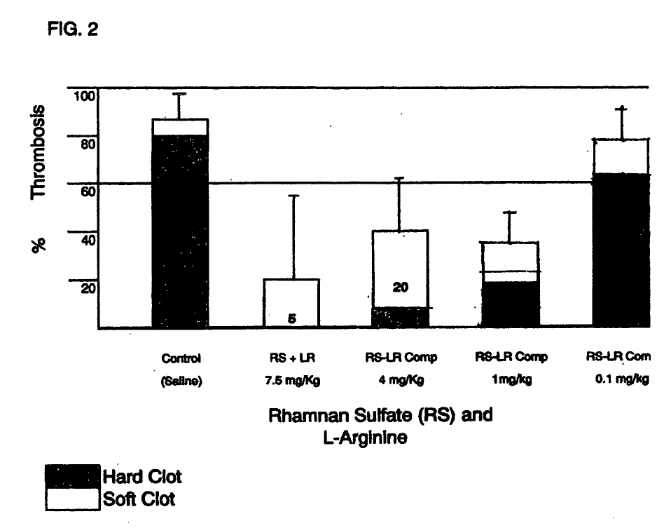

FIGS. 1 and 2 show anti-thrombotic activity of orally administered rhamnan sulphate or rhamnan sulphate and arginine as compared to oral unfractionated heparin. Error bars show 95% confidence intervals; upward bars for total clots, downward for hard clots. RS+LR is 7.5 mg/kg rhamnan sulphate+112.5 mg/kg arginine); RS-LR is 7.5 mg/kg rhamnan sulphate arginine complex. Numbers in bars show number of rats per group.

Plasma Levels

The Rhamnan Sulphate Groups at all doses did not have a significant effect on APTT or the Heptest (Table 2). Rhamnan sulphate alone or when complexed with arginine had little or no effect on anti-Xa or anti-Ila activity. Rhamnan sulphate alone had somewhat more anti-Xa activity than the rhamnan sulphate-arginine complex. When anti-Xa activity was measured in the plasma of rats there was a reduced optical density in the plasma samples from some of the rats given rhamnan or the rhamnan sulphate-arginine complex. (Data not shown). There was no evidence of bleeding or blood loss in the animals.

TABLE 3
Activation partial thromboplastin time and Heptest following oral
administration of rhamnan sulphate alone, with arginine or as a rhamnan
sulphate-arginine complex.

| | Dose | APTT (sec) | | Heptest (sec) | |
|---|---|---|---|---|---|
| | mg/kg | Mean | SE | Mean | SE |
| | Controls | 19.5 | 0.9 | 36.3 | 0.8 |
| Rhamnan | 7.5 | 20.2 | 0.6 | 32.5 | 2.2 |
| Sulphate | 4 | 20.5 | 0.5 | 34.2 | 1.9 |
| | 2 | 21.8 | 0.9 | 36.5 | 1.1 |
| Rhamnan sulphate + LR | 7.5 | 20.4 | 0.8 | 31.8 | 0.9 |
| Rhamnan Sulphate- | 4 | 22.6 | 1.5 | 36.9 | 1.0 |
| Arginine | 1 | 18.5 | 0.7 | 30.8 | 0.7 |
| Complex | 0.1 | 18.5 | 0.7 | 30.8 | 1.0 |

Rhamnan sulphate like material was also found on both aortic and vena caval endothelium. A higher concentration was found on the vena cava than on the aorta when all compounds were administered (Table 3) P<0.00003 one-tailed t-test. A dose effect was evident when venal caval concentrations of rhamnan sulphate were observed following rhamnan sulphate or rhamnan sulphate-arginine. A similar dose effect was seen for aortic concentrations of rhamnan sulphate following oral administration of rhamnan sulphate arginine but not rhamnan sulphate alone. Vena caval but not aortic concentrations were greater at 4 mg/kg for rhamnan sulphate but not rhamnan sulphate—arginine complex.

TABLE 4
Rhamnan sulphate - like material found on aortic and vena caval
endothelium following oral administration of rhamnan sulphate
alone, with arginine or as a rhamnan sulphate-arginine complex.

| | | | Aorta | Vena Cava |
|---|---|---|---|---|
| | Dose | | μg/cm² | μg/cm² |
| | mg/kg | Number | mean ± se | mean ± se |
| Rhamnan | 7.5 | 5 | 1.80 ± 0.79 | 11.22 ± 3.20 |
| Sulphate | 4 | 20 | 2.05 ± 0.08 | 15.97 ± 1.54* |
| | 2 | 5 | 2.32 ± 0.33 | 3.00 ± 0.43 |
| Rhamnan sulphate + | 7.5 | 5 | 3.91 ± 0.65 | 7.16 ± 3.77 |
| LR | | | | |
| Rhamnan Sulphate- | 4 | 20 | 2.30 ± 0.07 | 4.41 ± 0.05 |
| Arginine | 1 | 20 | 0.12 ± 0.05 | 0.42 ± 0.10 |
| Complex | 0.1 | 20 | 0.52 ± 0.13 | 1.89 ± 0.43 |

Rhamnan sulphate-like material was also recovered from the urine and feces accumulated over the 4 hr period. The amounts and concentrations recovered after administration of rhamnan sulphate alone resulted in more being excreted in the urine than when given as a rhamnan sulphate-arginine complex. Amounts recovered were 3.0±0.4 and 1.6±0.4 (mean±SE) percent of dose for rhamnan sulphate alone versus rhamnan sulphate-arginine respectively.

Amounts recovered from feces also show that more is recovered when administered as rhamnan sulphate alone versus rhamnan sulphate-arginine. A dose effect was evident. Amounts recovered were 13.7±4.4 and 6.1±1.9 (mean±SE) percent of dose for rhamnan sulphate alone versus rhamnan sulphate-arginine respectively, these differences were not significant.

In general, the results indicate that Rhamnan Sulphate provides vessel surface anti-thrombotic activity without appreciably increasing plasma anticoagulation activity. Hard clots and soft clots build from the inside surface of the lumen of the injured vessel and extend radially more central into the lumen of the vessel, but there is little or no change in the plasma coagulation activity as was measured by the standard plasma coagulation tests mentioned above. Thus, Rhamnan Sulphate is effective in preventing clot formation at the inside surface of the vessel, but it does not provide the patient with increased plasma anti-coagulation activity to render the patient a “bleeder” or to be at appreciably increased risk of hemorrhaging.

The results show that without any treatment, as a control group, saline had no effect on thrombosis with approximately a 90% incidence of thrombosis of which a very high percentage were hard clots. Heparin, which is commonly used as an anticoagulant, at 7.5 mg/kg showed little or no effect in total incidence of thrombosis, however it reduced the percentage of incidence of hard clots. Conversely, Rhamnan Sulphate at the same 7.5 mg/kg dosage showed a significant decrease in incidence of thrombosis with little or no hard clots. Reduction of the dosage of Rhamnan Sulphate to 4 mg/kg and 2 mg/kg resulted in the increase in incidence of thrombosis and in the re-appearance of hard clots from the 7.5 mg/kg dose.

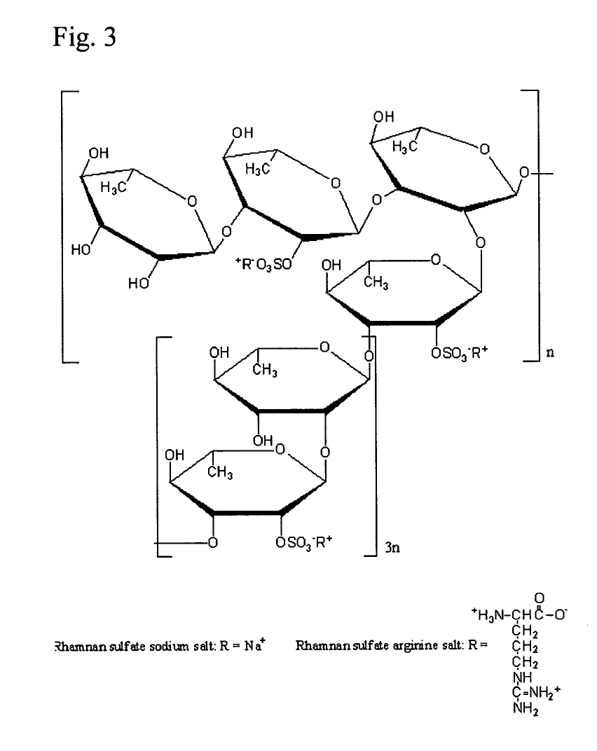

FIG. 2 again compares the control group with the co-administration of Rhamnan Sulphate with L-Arginine, the structure of which is commonly known. RS+LR refers to the co-administration of Rhamnan Sulphate, whereas RS-LR refers to the salt of Rhamnan Sulphate with arginine as a compound, the chemical structure of which is shown in FIG. 3. Group 6, RS+LR at 7.5 mg/kg, showed little difference with administration of Rhamnan Sulphate by itself both in the incidence of thrombosis and in the non-occurring of hard clots. Group 7, RS-LR compound at 4 mg/kg, however, showed a significant reduction in the total incidence of thrombosis from 4 mg/kg of Rhamnan Sulphate alone and a reduction in hard clots. Even Group 8, LS-LR compound at 1 mg/kg showed a slight decrease in incidence of thrombosis than and hard clots than 4 mg/kg of Rhamnan Sulphate.

The result is that Rhamnan Sulphate by itself is more effective than Heparin in lowering the incidence of thrombosis and in reducing the number of hard clots. Further, that the blood anticoagulation activity is not appreciably increased. This further desired effect is opposite that of Heparin, which is known to increase plasma anticoagulation activity. Thus, the use of Rhamnan Sulphate in treatment of endothelial dysfunction, particularly cardiovascular disease, and more particularly atheresclerosis and arteriosclerosis is desired. A second result is that the salt of Rhamnan Sulphate-arginine compound is more effective in lowering the incidence of thrombosis and hard clots than an equivalent dose of Rhamnan Sulphate alone.

### EXAMPLE 3

### Clinical Studies

Cardiovascular Risk Biomarkers

An open-label, multiple administration, dose escalation study was conducted to assess the influence of the compound of seaweed extract and L-arginine on vascular function and accepted plasma biochemical markers indicative of endothelial and cardiovascular function.

Method:

Eight healthy human subjects (HS), 8 men, aged 39.9±4.0 yrs, eight essential hypertension patients (HT), 5 men and 3 women, aged 57.6±4.4 yrs, and eight peripheral arterial occlusive disease patients (PAOD), 5 men and 3 women, aged 55.6±5.1 years, received three different amounts of proprietary compound (300, 600 and 1200 mg) each over 7 days as an oral dose after 6 hours fasting.

Results and Biochemical Markers:

Selected markers were identified to be relevant to cardiovascular risk assessment; high sensitive C-Reactive Protein (hs-CRP), HDL-cholesterol, total cholesterol, LDL-cholesterol, triglycerides, and blood pressure. These were part of a larger group of laboratory values.

Interpretation of these results in human subjects in light of authoritative published studies indicates significant vasoprotective effects from the administration of the proprietary seaweed extract powder and L-arginine compound.

hs-CRP was above the normal range in all hypertensive and all PAOD subjects, and in several healthy subjects. At baseline and at the end of the study (three weeks) the mean concentration of hs-CRP was lower in the healthy group as compared to the hypertensive and PAOD groups.

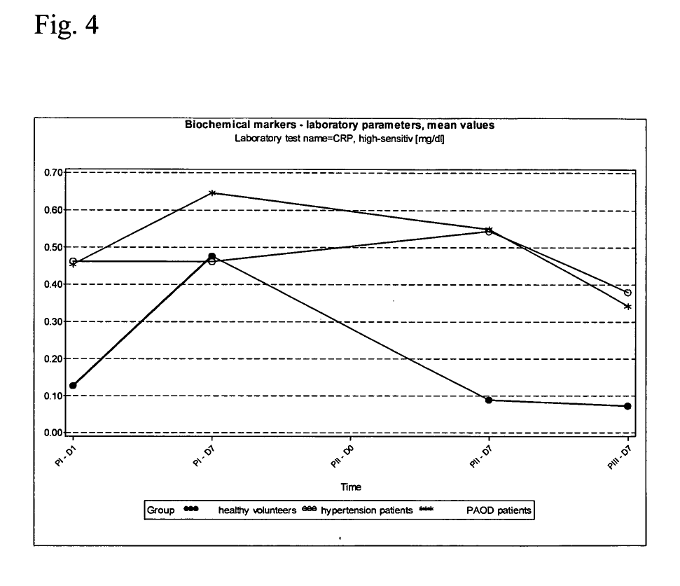

As is shown in FIG. 4, all groups demonstrated a decrease in hs-CRP from baseline to the end of the study. At all quartiles of hs-CRP there is a correlation with increased cardiovascular events regardless of LDL cholesterol level.

87% of healthy subjects demonstrated reductions in hs-CRP (up to 43% reduction) during the study. 75% of hypertensive subjects and 62% of PAOD subjects showed reduction of hs-CRP during the study. 80% of all patients in the study with hs-CRP levels greater than 3 times normal reduced their levels during the study. Those with the highest levels showed reductions up to 70% to the baseline values.

The literature suggests that a 30-40% reduction in hs-CRP indicates a reduction in risk of heart attack and stroke risk by at least 30%.

TABLE 5
CRP Test Results
Laboratory test name = CRP, high-sensitiv [mg/dl]

| | n | Mean | Median | SD |
|---|---|---|---|---|
| healthy volunteers | | | | |
| PI - D1 | 8 | 0.127 | 0.069 | 0.145 |
| PI - D7 | 8 | 0.476 | 0.111 | 0.741 |
| PII - D7 | 8 | 0.089 | 0.086 | 0.069 |
| PIII - D7 | 8 | 0.072 | 0.057 | 0.058 |
| hypertension patients | | | | |
| PI - D1 | 8 | 0.463 | 0.204 | 0.673 |
| PI - D7 | 8 | 0.462 | 0.327 | 0.435 |
| PII - D7 | 8 | 0.544 | 0.340 | 0.512 |
| PIII - D7 | 8 | 0.380 | 0.363 | 0.239 |
| PAOD patients | | | | |
| PI - D1 | 8 | 0.454 | 0.248 | 0.538 |
| PI - D7 | 8 | 0.647 | 0.466 | 0.807 |
| PII - D7 | 8 | 0.550 | 0.415 | 0.510 |
| PIII - D7 | 8 | 0.342 | 0.183 | 0.422 |
| Total | | | | |
| PI - D1 | 24 | 0.348 | 0.142 | 0.508 |
| PI - D7 | 24 | 0.528 | 0.295 | 0.656 |
| PII - D7 | 24 | 0.394 | 0.208 | 0.458 |
| PIII - D7 | 24 | 0.265 | 0.142 | 0.304 |

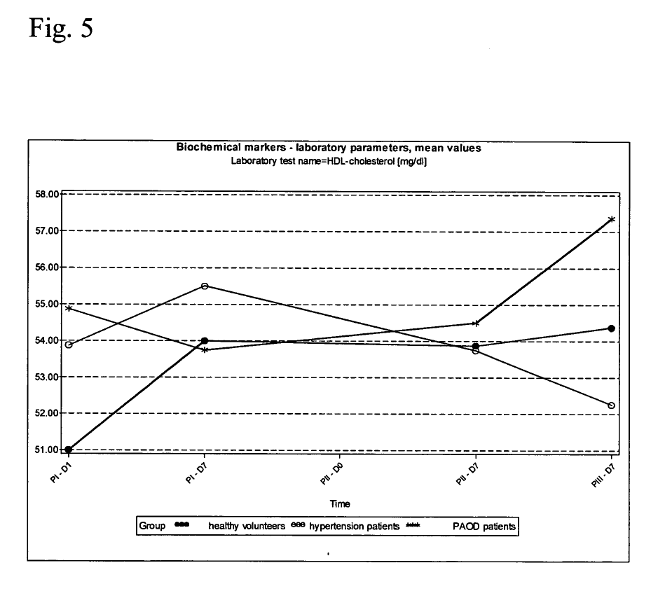

For HDL-cholesterol almost all subject groups were within the normal range; however, increases were noted in each subject group. All healthy subjects showed elevated HDL levels during the study compared with baseline values. HDL levels were observed to be as much as 38% increased in the healthy subjects within 7 days during the study. Half of hypertensive subjects showed increased HDL levels (up to 28% increase) during the study. Half of PAOD subjects showed increased HDL levels (up to 15%) during the study. As is shown in FIG. 5, the study results show that the combination of seaweed extract and the nitrogen donor Arginine, raised HDL by 7-8 mg/dl. The literature indicates that every 1% elevation in HDL reduces the risk of an adverse coronary event such as a heart attack, by 3%, which indicates that risk is reduced by another 24%.

For total cholesterol the majority of all measurements in all groups were above the normal range throughout the study. The healthy and POAD groups showed a mean increase through the second period, then a slight drop to the end of the third period. The hypertensive group showed a slight mean increase through the first period, followed by decreases in the second and third periods, resulting in a drop of 9% from the end of the first period to the end of the third period. The hypertensive group ended lower than the POAD group at the end of period three.

TABLE 6
HDL Test Results
Laboratory test name = HDL-cholesterol [mg/dl]

| | n | Mean | Median | SD |
|---|---|---|---|---|
| healthy volunteers | | | | |
| PI - D1 | 8 | 51.00 | 53.50 | 9.94 |
| PI - D7 | 8 | 54.00 | 52.00 | 10.73 |
| PII - D7 | 8 | 53.88 | 52.00 | 11.73 |
| PIII - D7 | 8 | 54.38 | 54.00 | 9.49 |
| hypertension patients | | | | |
| PI - D1 | 8 | 53.88 | 56.00 | 12.93 |
| PI - D7 | 8 | 55.50 | 54.00 | 12.67 |
| PII - D7 | 8 | 53.75 | 54.00 | 10.75 |
| PIII - D7 | 8 | 52.25 | 53.00 | 7.67 |
| PAOD patients | | | | |
| PI - D1 | 8 | 54.88 | 52.50 | 5.06 |
| PI - D7 | 8 | 53.75 | 51.50 | 7.80 |
| PII - D7 | 8 | 54.50 | 52.50 | 11.40 |
| PIII - D7 | 8 | 67.38 | 51.00 | 12.36 |
| Total | | | | |
| PI - D1 | 24 | 53.25 | 53.00 | 9.57 |
| PI - D7 | 24 | 54.42 | 53.00 | 10.23 |
| PII - D7 | 24 | 54.04 | 53.00 | 10.80 |
| PIII - D7 | 24 | 54.67 | 52.50 | 9.82 |

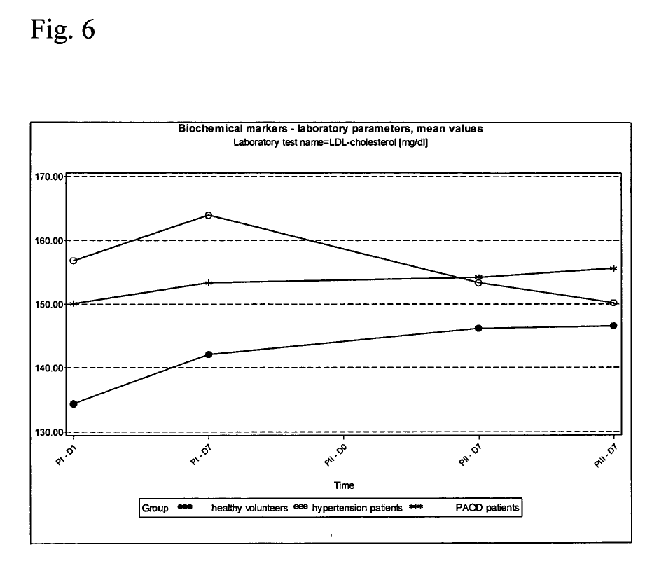

For LDL-cholesterol approximately half of all measurements in all subjects were above the normal range throughout the study. The healthy and PAOD groups showed a slight mean increase throughout the study. As reflected in the total cholesterol results, as is shown in FIG. 6, the hypertensive group showed an increase through the first period, followed by decreases in the second and third periods, resulting in a drop of 9% from the end of the first period to the end of the third period. The hypertensive group ended lower than the POAD group at the end of period three.

TABLE 7
LDL Test Results
Laboratory test name = LDL-cholesterol [mg/dl]

| | n | Mean | Median | SD |
|---|---|---|---|---|
| healthy volunteers | | | | |
| PI - D1 | 8 | 134.38 | 141.50 | 24.77 |
| PI - D7 | 8 | 142.13 | 145.00 | 37.01 |
| PII - D7 | 8 | 146.25 | 154.00 | 28.04 |
| PIII - D7 | 8 | 146.63 | 150.50 | 31.42 |
| hypertension patients | | | | |
| PI - D1 | 8 | 156.88 | 154.00 | 31.15 |
| PI - D7 | 8 | 164.00 | 159.50 | 32.32 |
| PII - D7 | 8 | 153.38 | 151.00 | 23.21 |
| PIII - D7 | 8 | 150.25 | 151.00 | 26.67 |
| PAOD patients | | | | |
| PI - D1 | 8 | 150.13 | 151.00 | 16.24 |
| PI - D7 | 8 | 153.38 | 152.50 | 28.92 |
| PII - D7 | 8 | 154.25 | 145.50 | 24.31 |
| PIII - D7 | 8 | 155.63 | 149.50 | 31.30 |
| Total | | | | |
| PI - D1 | 24 | 147.13 | 148.00 | 25.59 |
| PI - D7 | 24 | 153.17 | 152.00 | 32.75 |
| PI - D7 | 24 | 151.29 | 150.00 | 24.42 |
| PIII - D7 | 24 | 150.83 | 150.50 | 28.80 |

Growing evidence indicates that hs-CRP levels are a stronger predictor of cardiovascular events than LDL levels. However, because hs-CRP and LDL measurements tend to identify different high-risk groups, screening for both biologic markers appears to provide better prognostic information than screening for either marker alone.

For triglycerides almost all measurements in the healthy subjects were within the normal range throughout the study and almost all measurements in the hypertensive subjects were above the normal range. In the PAOD subject group approximately half of all measurements were above the normal range throughout the study.

TABLE 8
Total triglycerides
Laboratory test name = total triglycerides [mg/dl]

| | n | Mean | Median | SD |
|---|---|---|---|---|
| healthy volunteers | | | | |
| PI - D1 | 8 | 109.13 | 110.00 | 52.65 |
| PI - D1 | 8 | 90.00 | 83.50 | 40.92 |
| PII - D7 | 8 | 132.38 | 136.00 | 42.94 |
| PIII - D7 | 8 | 107.50 | 110.00 | 33.30 |
| hypertenaion patients | | | | |
| PI - D1 | 8 | 279.63 | 221.50 | 209.57 |
| PI - D7 | 8 | 230.38 | 216.50 | 88.94 |
| PII - D7 | 8 | 245.75 | 210.00 | 137.67 |
| PIII - D7 | 8 | 249.88 | 260.00 | 93.97 |
| PAOD patients | | | | |
| PI - D1 | 8 | 176.50 | 133.50 | 98.23 |
| PI - D1 | 8 | 161.88 | 145.50 | 92.57 |
| PII - D7 | 8 | 160.88 | 148.00 | 75.46 |
| PIII - D7 | 8 | 194.00 | 108.00 | 153.51 |
| Total | | | | |
| PI - D1 | 24 | 188.42 | 135.50 | 149.25 |
| PI - D7 | 24 | 160.75 | 134.50 | 94.62 |
| PII - D7 | 24 | 179.67 | 153.50 | 102.36 |
| PIII - D7 | 24 | 183.79 | 121.50 | 117.38 |

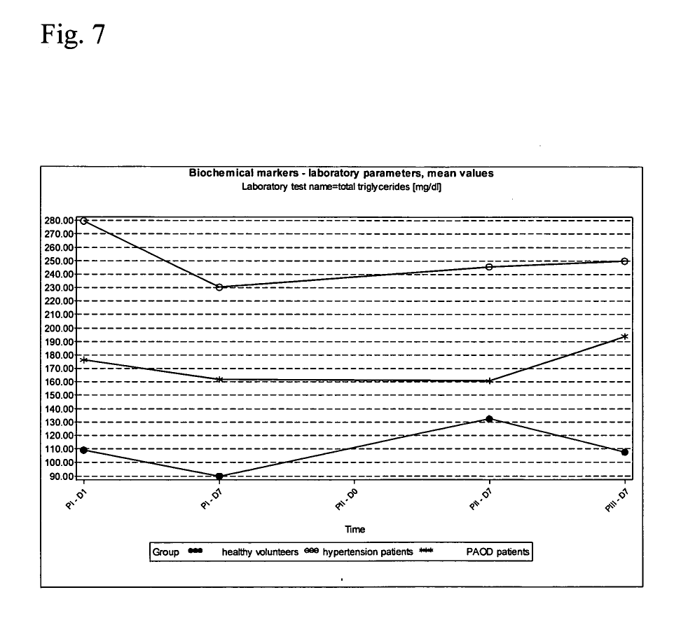

As is shown in FIG. 7, each subject group demonstrated a mean decrease at the end of the first period: the healthy subjects at −18%, the hypertensives at −18%, and the PAOD at −8%. The healthy and hypertensive subjects demonstrated a decrease at the end of period three compared to baseline, healthy subjects at −2% and hypertensive subjects at −11%. The PAOD demonstrated an increase at the end of period three compared to baseline of +10%. The study indicated that the compound lowers triglycerides by 50 mg/dl. This would indicate that it yielded a risk reduction of at least 20%.

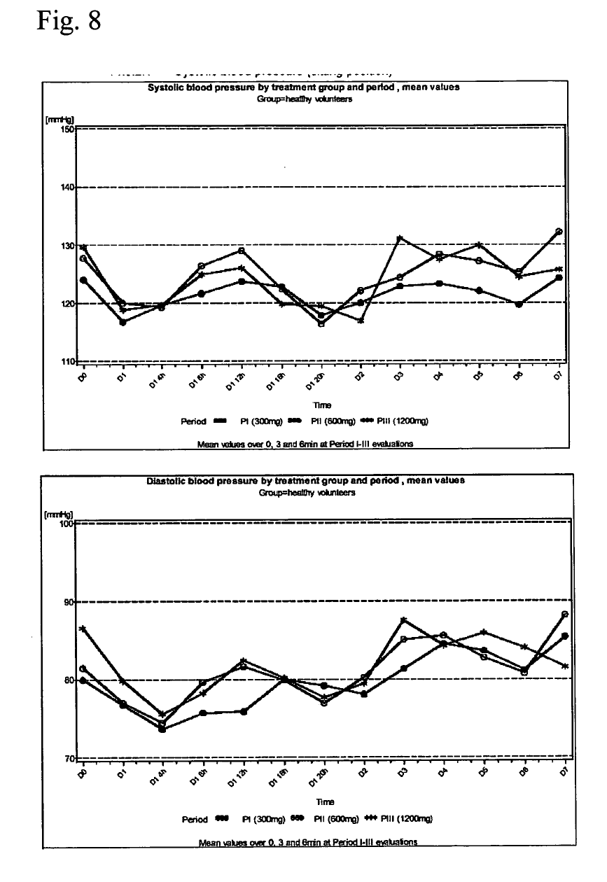

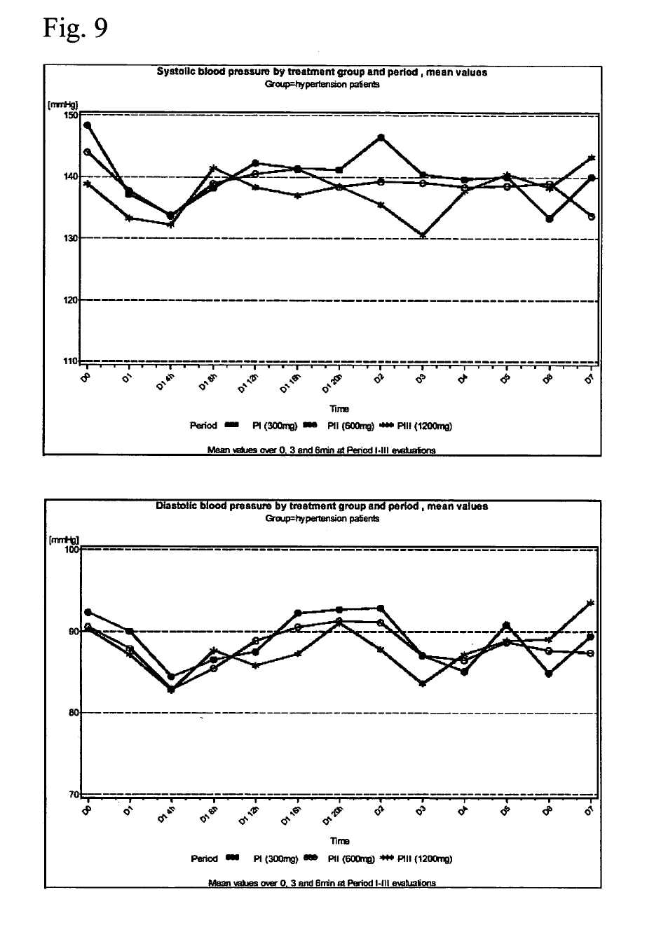

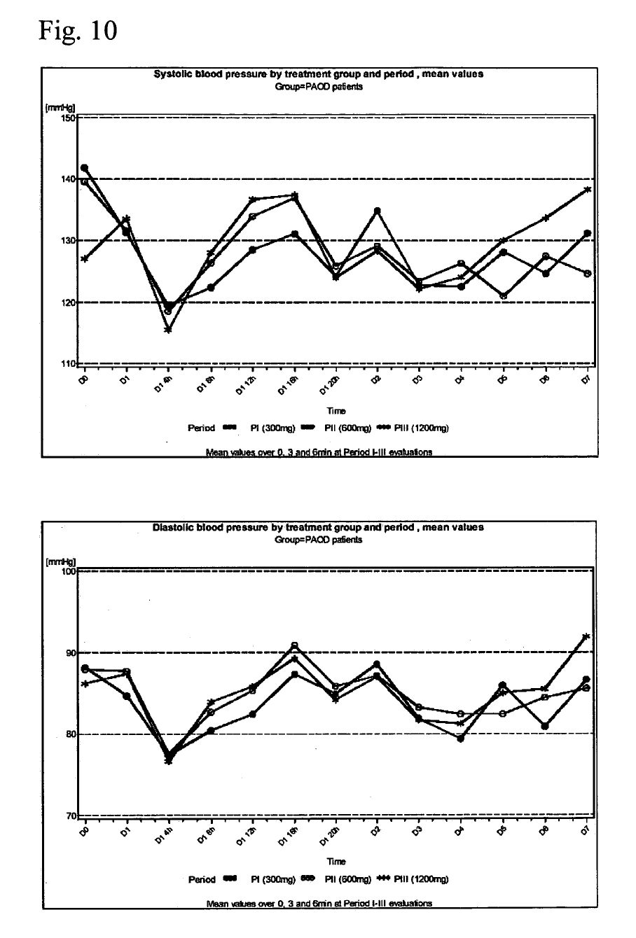

Vasodilation is the state of normalcy in arteries and minimizes flow-stress injury in arteries and abnormal smooth muscle growth. Vasodilation induced by the proprietary compound of seaweed extract and L-arginine reduced mean systolic BP up to 11 mm Hg following administration in the healthy tested subjects, 15 mmHg in the hypertensives, and 22 mmHg in the PAOD subject group. Mean diastolic BP was reduced up to 11 mm Hg following administration in the healthy tested subjects, 8 mmHg in the hypertensives, and 11 mmHg in the PAOD subject group. All study participants demonstrated vasodilation as evidenced by BP reduction, typically by 10-20 mm. For every 20 mm reduction of BP, risk of an adverse cardiovascular event reduces by 50%. The above is shown in FIGS. 8, 9, and 10.

These vasoprotective effects are mutually reinforcing and considered additive to the 30-35% seen with statin drug administration.

Mechanism of Action:

While not being bound to a mechanism of action, it is thought that the compound of seaweed extract and L-arginine is an endothelial modulating agent due to its direct uptake and preferential binding to endothelial cells.

As evidenced by the results in normal human subjects, enhancement of endothelial function with the administration of the compound of seaweed extract and L-arginine, prior to overt disease, reinforces the normal vasoprotective functions of the endothelium and preserves normal vascular function.

It is therefore evident how the objective of the present invention is satisfied. First the method and composition of the invention possesses extremely potent anti-thrombotic activity and other inhibitory effects on cell surface coagulation assembly and activity for thrombus inhibition. It is well understood that accelerated cardiovascular arteriosclerosis, hypertension, diabetic nephropathy, and congestive heart failure are long-term complications of the diabetic processes. Each of these disease states is accompanied by decreased eNOS activity, increased free radical generation, increased thrombogenic activity and therefore thrombotic and ischemic complications.

Second, since the seaweed extract is from plant cells, it has no potential for the transmission of potentially lethal and serious prion diseases such as mad cow disease.

Third, the seaweed extract has no potential for activating Platelet Factor IV and resulting in immune complex destruction of platelets as seen with heparin administration.

Fourth, the seaweed extract is a functional substitute for heparin in applications requiring systemic (not Plasma) anticoagulant activity such as dialysis, bypass surgery, and polymer tube coatings and devices for use in mammals and humans.

Fifth, the seaweed extract composition has less peptide residues because it is extracted from plant cells as compared to heparin from animal cells. Hence, it is less allergic reaction prone and has fewer immunogenic properties.

Sixth, the seaweed extract of the present invention reduces C-Reactive Protein levels in the plasma.

Seventh, the seaweed extract of the present invention to preserves healthy cardiovascular function including blood vessels, by lowering LDL and/or increasing HDL level in the plasma.

It will be readily apparent to those skilled in the art that many modifications, derivations and improvements are within the scope of the invention. Such modifications, derivations, and improvements should be accorded full scope of protection by the claims appended hereto.

1. A method for treatment of Type II Diabetes Mellitus and its complications consisting neuropathy, nephropathy, arteriosclerosis, and proterinuria comprising the step of administering to a mammal an amount of seaweed extract in effective doses.

2. The method of claim 1 wherein the seaweed is selected from the group consisting of brown algae, red algae and green algae.

3. The method of claim 1 wherein the seaweed extract contains a polysaccharide.

4. The method of claim 3 wherein the polysaccharide is selected from the group consisting of; Rhamnose, Xylose, Galactose, and Mannose, either individually or a combination thereof.

5. The method of claim 1 wherein the seaweed extract contains between approximately 63 mole % to 78 mole % Rhamnose.

6. The method of claim 1 wherein the seaweed extract contains between approximately 6.5 mole % to 9.2 mole % Xylose.

7. The method of claim 1 wherein Rhamnose and Xylose are present in the seaweed extract in amounts between a ratio of approximately 12 Rhamnose to 1 Xylose and approximately 8 Rhamnose to 1 Xylose.

8. The method of claim 2 wherein the brown algae is selected from the group consisting of Fucus vesiculosus, Fucus Evanescens, Laminaria brasiliensis, or Ascophylum nodosum.

9. The method of claim 2 wherein the green algae is selected from the group consisting of Monostroma nitidium, Monostroma zosteticola, Monostroma angicava, Monostroma lattissimum, Monostroma pulchrum, Monostroma fusem, Monostroma grevillei, Entoromorpha compressa, Ulva arasakii, Ulva Pertussa, Cladophora denna, Cladophora rugulosa, Chaecomorpha spiralis, Chaecomorpha crassa, Spongomorpha duriuscula, Codium fragile, Codium divaricaium Codium latum, or Caulerpa okamarai.

10. The method of claim 1 wherein the Type II Diabetes Mellitus is caused by endothelial dysfunction.

11. The method of claim 1 further comprising the co-administration of a nitrogen donor together with the seaweed extract.

12. The method of claim 11 wherein the nitrogen donor is selected from the group consisting of L-Arginine and Lysine.

13. The method of claim 1 wherein the seaweed extract is administered orally.

14. The method of claim 1, wherein the seaweed extract is mixed with food stuffs; wherein the food stuffs is selected from the group consisting of cereals, bread, drinks, health bars, juices, concentrates, canned food, ice cream, water, staple goods such as wheat, corn, barley, and oat in any form, or taste maskers such as sugar or ascorbic acid.

15. The method of claim 1 wherein a daily dose of seaweed extract is equivalent to between approximately 2,000 IU and 200,000 IU of heparin activity.

16. The method of claim 1 wherein a daily dose of seaweed extract is equivalent to between approximately 5,000 IU and 20,000 IU of heparin activity.

17. The method of claim 1 wherein a daily dose of seaweed extract is equivalent to between approximately 8,000 IU and 12,000 IU of heparin activity.

18. The method of claim 1 wherein a daily dose of seaweed extract is approximately 7.5 mg/kg.

19. The method of claim 1 wherein the dose of seaweed extract is repeated.

20. The method of claim 1 wherein the anticoagulation activity in the blood plasma of the mammal is not appreciably increased.
